(19) United States
(12) Patent Application Publication
(10) Pub. No.: US 2012/0088721 A1
(43) Pub. Date: Apr. 12, 2012

(54) Cartilage Repair

(75) Inventors: Aviva Shiedlin, Brookline, MA (US); Timothy J. Butler, Malden, MA (US); Michael Philbrook, Boston, MA (US); Peter K. Jarrett, Lexington, MA (US)

(73) Assignee: GENZYME CORPORATION, Cambridge, MA (US)

(21) Appl. No.: 13/258,054

(22) PCT Filed: Mar. 22, 2010
(86) PCT No.: PCT/US2010/028086

§ 371 Date: Nov. 30, 2011

Related U.S. Application Data
(60) Provisional application No. 61/162,447, filed on Mar. 23, 2009.

(51) Int. Cl. A61K 31/728; A61K 38/30; B29C 31/00; A61K 38/18

(52) U.S. Cl. 514/8.6; 514/54; 514/8.8; 514/8.9; 514/8.5; 264/122

(57) ABSTRACT

This invention relates to compositions, methods of preparation thereof, and use thereof for cartilage repair.

### TECHNICAL FIELD

This invention relates to compositions, methods of preparation thereof, and use thereof for cartilage repair.

### BACKGROUND

Cartilage damage is common in humans. If untreated, the damage can progressively worsen and can lead to chronic conditions such as osteoarthritis. A number of different therapeutic methods are currently being used to repair damaged cartilage. Exemplary methods include implantation of chondrocytes or mesenchymal stem cells directly or via a cell delivery vehicle into the osteochondral defect, or using growth factors to promote the repair processes (Gao, et al. Clinical Orthopaedics and Related Research 2004, S62-66). Durability of the repair tissue, certainty of the initial optimal growth factor dosage, or knowledge of the interaction among multiple biofactors are important and sometimes problematic (Gao, et al. Clinical Orthopaedics and Related Research 2004, S62-66).

### SUMMARY

We have discovered certain compositions and methods for repairing cartilage.

In one aspect, the invention features a formable composition including from about 25% to about 40% by weight of demineralized bone matrix (DBM), from about 3.5% to about 25% by weight of a hyaluronic acid salt, and from about 40% to about 72% by weight of a biologically compatible liquid

In some embodiments, the hyaluronic acid salt can be a sodium salt, potassium salt, or calcium salt of hyaluronic acid.

In some embodiments, the formable composition can include a biologically compatible liquid, which can be PBS, water, saline, or LRS, for example, PBS.

The formable composition can further comprise a rheology modifier, for example, glycerol or carboxymethyl cellulose.

In some embodiments, the formable composition can include less than 35% by weight of demineralized bone matrix, for example, from about 25% to about 35% by weight of demineralized bone matrix.

In some embodiments, the formable composition can comprise at least about 6% by weight of the hyaluronic acid salt. In certain embodiments, the formable composition can comprise about 6% to about 15% by weight of the hyaluronic acid salt. In other embodiments, the formable composition can comprise about 6% to about 9% by weight of the hyaluronic acid salt.

In some embodiments, the hyaluronic acid salt can have an average molecular weight of at least about 500,000 Daltons.

In some embodiments, the formable composition can comprise a mixture of at least two hyaluronic acid salts. In other embodiments, the formable composition can comprise a mixture of two hyaluronic acid salts, e.g., a hyaluronic acid salt of a first average molecular weight in a range of about 0.05 to about 1.0 million Daltons and a hyaluronic acid salt of a second average molecular weight in a range of about 1.0 to about 5.0 million Daltons. The average molecular weight ratio of the hyaluronic acid salt of the first average molecular weight to the hyaluronic acid salt of the second average molecular weight can range from 1:5 to 5:1.

In some embodiments, the average molecular weight difference between the hyaluronic acid salt of the first average molecular weight and the hyaluronic acid salt of the second average molecular weight can be at least about 0.5 million Daltons.

In some embodiments, the formable composition can be further characterized in test. For example, when subject to the Instron Syringe Extrusion Force (ISEF) test, the composition can exhibit an extrusion force of from about 12.0 Newtons to about 30.0 Newtons. In some embodiments, the extrusion force can be from about 18.0 Newtons to about 26.0 Newtons. In embodiments, the extrusion force is measured in a 3 mL syringe with a diameter of 8.6 mm and 15 gauge 1-1/1 needle.

The formable composition can further comprise a pharmaceutically active ingredient. The pharmaceutically active ingredient can be bone morphogenic protein, tissue growth factors, insulin growth factors, antioxidants, antibiotics, or combinations of growth factors. In embodiments, the pharmaceutically active ingredient can be selected from BMP-2, BMP-4, BMP-6, BMP-7, TGF-B, IGF-1, ascorbate, pyruvate, BHT, gentamycin, vancomycin, the combination of TGF-β and BMP-2, and the combination of TGF-β and IGF-1.

In some embodiments, the pharmaceutically active ingredient can be conjugated with the hyaluronic acid salt.

In another aspect, this invention features a dry blend composition comprising from about 60% to about 92% by weight of demineralized bone matrix, from about 3.5% to about 38% by weight of a hyaluronic acid salt, and from about 3% to about 10% by weight of a biologically compatible liquid. In some embodiments, the hyaluronic acid salt has an average molecular weight of at least about 200,000 Daltons.

In still another aspect, this invention features a plug comprising from about 25% to about 88% by weight of demineralized bone matrix and from about 3.5% to about 38% by weight of a hyaluronic acid salt and from about 3% to about 20% by weight of a biologically compatible liquid. In some embodiments, the composition includes from about 5% to about 10% by weight of a biologically compatible liquid. In some embodiments, the hyaluronic acid salt has an average molecular weight of at least about 200,000 Daltons. In some embodiments, the plug comprises less than about 10% of a biological liquid. In certain embodiments, the plug comprises about or less than about 5% by weight of a biological liquid. In some embodiments, the biologically compatible liquid can be water. For example, the biological compatible liquid is residual moisture that can be measured by loss on drying. In some embodiments, the plug exhibits an unconfined Compression Stress of at least about 1.55 MPa (e.g., at least about 1.60 MPa, or at least about 1.65 MPa, or at least about 1.70 MPa). In certain embodiments, the plug exhibits an unconfined Compression Stress of from about 1.55 MPa to about 1.70 MPa.

In one aspect, this invention features a method of repairing cartilage in a subject comprising administering to a subject at a site of a defect in cartilaginous tissue an effective amount of a composition, the composition comprising demineralized bone matrix and a hyaluronic acid salt. The composition can be a formable composition, a dry blend composition, or a plug.

In other aspect, this invention features a method of preparing a formable composition, the composition comprising from about 25% to about 40% by weight of demineralized bone matrix, from about 3.5% to about 25% by weight of a hyaluronic acid salt, and from about 40% to about 72% by weight of a biologically compatible liquid, the method comprising mixing solid components, followed by the addition of a liquid component. The solid components can include demineralized bone matrix and a hyaluronic acid salt. The hyaluronic acid salt can have an average molecular weight of at least about 200,000 Daltons. In some embodiments, the liquid component can be added before the intended use. In some embodiments, the liquid component is water. In cases where the composition comprises a pharmaceutically active ingredient, the pharmaceutically active ingredient can be conjugated with the hyaluronic acid salt. In other embodiments, the pharmaceutically active ingredient can be mixed with the solid components, such as the demineralized bone matrix and the hyaluronic acid salt. In certain embodiments, the pharmaceutically active ingredient can be mixed with the liquid component.

In still another aspect, this invention features a method of forming a plug comprising

a) providing powders of demineralized bone matrix and a hyaluronic acid salt,

b) mixing the powders,

c) adding a liquid component to form a putty like material,

d) placing the putty in a mold, and

e) drying the shaped putty to form a plug.

Generally “putty” is firm yet pliable. It does not crumble. It has a malleable consistency that can be shaped by hand, or forced into bone voids or cancellous interstices with manual pressure.

In some embodiments, the powders can further comprise a plasticizer such as glycerol or PEG.

In some embodiments, the drying can be lyophilizing, air drying, or oven or vacuum drying.

In certain embodiments, the plug can be further conditioned to achieve a moisture content of about 3-20% by weight (e.g., at least 5% by weight). In some embodiments, the moisture content is from about 5% to about 10% by weight. In other embodiments, the moisture content is from about 5% to about 15% by weight.

As used herein, a “formable composition” is one that is capable of being manipulated by a surgeon to a desired shape substantially without adherence to the gloves, and consistent with good surgical technique. For example, the formable composition can be shaped to conform to the contours of the surgical defect. A putty may be one type of formable composition and may be used in a patient. More typically we use the term elsewhere in this application to describe an intermediate material that is dried into a plug. As used herein, “the average molecular weight” refers to the weight average molecular weight (Mw) that can be calculated by

Mw=ΣNi²Mi²/ΣNi Mi

where Ni is the number of molecules of molecular weight Mi.

As used herein, a “biologically compatible liquid” is one that is physiologically acceptable and does not cause unacceptable cellular injury. Examples of such liquids are water, buffers, saline, protein solutions, and sugar solutions.

As used herein, the term “subject” or “patient,” used interchangeably, refers to any animal, including mammals, preferably mice, rats, other rodents, rabbits, dogs, cats, swine, cattle, sheep, horses, or primates, and most preferably humans.

As used herein, the phrase “an effective amount” refers to the amount of active compound or pharmaceutical agent that elicits the biological or medicinal response that is being sought in a tissue, system, animal, individual or human by a researcher, veterinarian, medical doctor or other clinician.

As used here, the term “repair” is intended to mean without limitation repair, regeneration, reconstruction, reconstitution or bulking of tissues.

The details of one or more embodiments of the invention are set forth in the description below. Other features, objects, and advantages of the invention will be apparent from the description and from the claims.

### DETAILED DESCRIPTION

This invention is based, at least in part, on the unexpected discoveries that certain compositions can be used to repair cartilage.

Compositions

The compositions that can be used to repair cartilage include, but are not limited to a formable composition, a dry blend composition, and a plug.

A formable composition can include from about 20% to about 40% by weight of demineralized bone matrix (DBM), preferably from about 25% to about 40% or from about 25% to about 35%, more preferably from about 30% to about 40%, most preferably from about 30% to about 35%. The weight ratio between DBM and a hyaluronic acid salt can range from about 1:1 to about 25:1, or from about 2:1 to about 20:1, or from about 2:1 to about 15:1, or from about 2.5:1 to about 7.5:1.

The formable composition also include a biologically compatible liquid component, such as water or saline. In some embodiments, the liquid component can be Lactated Ringer's solution (LRS). In other embodiments, the liquid component includes physiologically acceptable buffered saline solutions such as phosphate buffered saline solutions (PBS).

In some embodiments, the formable composition of the present invention can include from about 40% to about 72% by weight of the liquid component (e.g., from about 45% to about 66%, or from about 50% to about 66%, or from about 60% to about 66%, or from about 62% to about 65%). In some embodiments, the liquid component is substantially free of organic solvent. Examples of the organic solvents include ethanol, isopropanol, N-methylpyrrolidone, propylene glycol, glycerol, low molecular weight polyethylene glycol, and DMSO.

The formable composition can include at least about 3.5% (e.g. at least about 5%, at least about 6%, at least about 9%, at least about 12%, or at least about 15%, or at least about 20% by weight of a hyaluronic acid salt. In some embodiments, the composition can include at least about 6% by weight of the hyaluronic acid salt.

In some embodiments, the formable composition can include from about 3.5% to about 25% (e.g., from about 5% to about 25%, from about 6% to about 25%, from about 9% to about 25%) by weight of the hyaluronic acid salt. In some embodiments, the formable composition can include from about 3.5% to about 20% (e.g., from about 5% to about 20%, from about 6% to about 20%, from about 9% to about 20%) by weight of the hyaluronic acid salt. In some embodiments, the formable composition includes from about 3.5% to about 15% (e.g., from about 5% to about 15%, from about 6% to about 15%, or from about 9% to about 15%) by weight of the hyaluronic acid salt. In some embodiments, the composition includes from about 3.5% to about 10% (e.g., from about 5% to about 10%, from about 6% to about 10%, or from about 9% to about 10%) by weight of the hyaluronic acid salt. In certain embodiments, the formable composition includes from about 3.5% to about 9% (e.g., from about 5% to about 9% or from about 6% to about 9%) by weight of the hyaluronic acid salt.

In some embodiments, the formable composition exhibits an extrusion force of from about 12.0 to about 30.0 Newtons subject to the Instron Syringe Extrusion Force (ISEF) test. For example, the formable composition can exhibits an extrusion force of from about 14.0 to about 26.0 Newtons (e.g., from about 14.0 to about 23.0 Newtons, or from about 14.0 to about 21.0 Newtons, or from about 14.0 to about 19.0 Newtons, or from about 14.0 to about 16.0 Newtons). In other embodiments, the formable composition can exhibit an extrusion force of from about 16.0 to about 26.0 Newtons (e.g., from about 18.0 to about 26.0 Newtons, or from about 20.0 to about 26.0 Newtons, or from about 22.0 to about 26.0 Newtons).

In some embodiments, the formable composition can further include a rheology modifier, for example, glycerol or carboxymethyl cellulose.

In some embodiments, the hyaluronic acid salt has an average molecular weight of at least about 500,000 Daltons (Da) (e.g., at least about 0.8 MDa, at least about 1.0 MDa, at least about 1.2 MDa, at least about 1.5 MDa, at least about 1.8 MDa, or at least about 2.0 MDa).

In cases where the compositions are dry blend compositions, a dry blend composition can include from about 60% to about 92% (e.g., from about 70% to about 92%, from about 80% to about 92%, from 85% to about 92%, or from about 90% to about 92%) by weight of DBM. In some embodiments, the dry blend composition can include from about 3.5% to about 38% (e.g., from about 4% to about 38%, from about 6% to about 38%, from about 9% to about 38%, from about 9% to about 30%, from about 9% to about 25%, from about 9% to about 20%) by weight of a hyaluronic acid salt. In embodiments, the dry blend composition can include from about 3% to about 10% (e.g., from about 3% to about 9%, from about 3% to about 7%, from about 3% to about 5%) by weight of residual moisture originating from both the DBM and the hyaluronic acid salt.

When the compositions are prepared as plugs, the amount of DBM in a plug can range from 25% to about 88% by weight of the total composition (e.g., from about 35% to about 88%, from about 55% to about 88%, from about 60% to about 88%, from about 65% to about 88%, from about 75% to about 88%, or from about 85% to about 88%).

The amount of the hyaluronic acid salt in a plug can range from about 3.5% to about 38% by weight of the total composition (e.g., from about 5% to about 38%, or from about 6% to about 38%, from about 6% to about 28%, from about 6% to about 24%, from about 6% to about 18%, or from about 6% to about 15%, or from about 7% to about 15%). In some embodiments, the weight of the hyaluronic acid salt is at least about 6% (e.g. at least about 7%, at least about 15%, or at least about 20%) by weight of the total composition. In some embodiments, the plug contains from about 3% to about 20% by weight of a biological compatible liquid, for example, less than 10% by weight. In some embodiments, the biologically compatible liquid can be water. For example, the biological compatible liquid is residual moisture that can be from about 3% to about 20% by weight (e.g., from about 5% to about 10%,from about 5% to about 15%). The residual moisture or other low volatile solvent may be measure by the loss on drying methods. In certain embodiments, the loss on drying is from about 3% to about 20% (e.g., from about 3% to about 17%, from about 5% to about 12%, from about 5% to about 10%).

A suitable particle size of DBM in a formable composition, a dry blend composition, and a plug can range from about 70 microns to about 850 microns, for example, from about 150 microns to about 800 microns or from 200 microns to about 800 microns. A suitable particle size of a hyaluronic acid salt can be about 600 microns, or about 500 microns, or about 400 microns.

In some embodiments, the hyaluronic acid salt in a formable composition has an average molecular weight of at least about 500,000 Daltons ((e.g., at least about 0.8 MDa, at least about 1.0 MDa, at least about 1.2 MDa, at least about 1.5 MDa, at least about 1.8 MDa, or at least about 2.0 MDa). In some embodiments, the hyaluronic acid salt in a dry composition or a plug can have an average molecular weight of at least about 200,000 Daltons (e.g., at least about 0.4 MDa, at least about 0.6 MDa, at least about 0.8 MDa, at least about 1.0 MDa, or at least about 1.2 MDa).

The compositions of the present invention can be formable compositions, dry blend compositions, or the plugs.

The compositions can include a mixture of at least two hyaluronic acid salts, for example, a mixture of two hyaluronic acid salts or a mixture of more than two (e.g., three or four) hyaluronic acid salts. In some embodiments, the compositions can include a mixture of two hyaluronic acid salts, comprising a hyaluronic acid salt of a first average molecular weight and a hyaluronic acid salt of a second average molecular weight. In some embodiments, the hyaluronic acid salt of the first average molecular weight is about 0.05-1.0 million Daltons (MDa) (e.g., about 0.05 MDa, about 0.1 MDa, about 0.3 MDa, about 0.6 MDa, about 0.8 MDa, or about 1.0 MDa).

In some embodiments, the hyaluronic acid salt of the first average molecular weight is about 0.1-1.0 million Daltons. In some embodiments, the hyaluronic acid salt of the first average molecular weight is about 0.3-1.0 million Daltons. In certain embodiments, the hyaluronic acid salt of the first average molecular weight is about 0.3-0.8 million Daltons, e.g., about 0.3-0.6 million Daltons.

In some embodiments, the hyaluronic acid salt of the second average molecular weight is about 1.0-5.0 million Daltons (MDa) (e.g., about 1.0 MDa, about 1.4 MDa, about 2.0 MDa, about 2.5 MDa, about 3.0 MDa, about 4.0 MDa, or about 5.0 MDa).

In some embodiments, the hyaluronic acid salt of the second average molecular weight is about 1.2-4.0 million Daltons. In certain embodiments, the hyaluronic acid salt of the second average molecular weight is about 1.0-3.0 million Daltons. For example, the hyaluronic acid salt of the second average molecular weight can be about 1.0-2.0 million Daltons, e.g., about 1.0-1.4 million Daltons.

The compositions of the present invention can include a mixture of two hyaluronic acid salts, comprising a hyaluronic acid salt of a first average molecular weight and a hyaluronic acid salt of a second average molecular weight. It is one aspect of the present invention that two hyaluronic acid salts in a composition allow for the manipulation of rheological properties of the composition.

In some embodiments, the hyaluronic acid salt of the first average molecular weight can be about 0.05-1.0 million Daltons and the hyaluronic acid salt of the second average molecular weight can be about 1.0-5.0 million Daltons. For example, the hyaluronic acid salt of the first average molecular weight can be about 0.1-1.0 million Daltons and the hyaluronic acid salt of the second average molecular weight can be about 1.0-3.0 million Daltons. In certain embodiments, the hyaluronic acid salt of the first average molecular weight can be 0.3-0.8 million Daltons and the hyaluronic acid salt of the second average molecular weight can be 1.0-2.0 million Daltons. In other embodiments, the hyaluronic acid salt of the first average molecular weight can be 0.3-0.6 million Daltons and the hyaluronic acid salt of the second average molecular weight can be about 1.0-1.6 million Daltons. For example, the hyaluronic acid salt of the first average molecular weight can be 0.4-0.6 million Daltons and the hyaluronic acid salt of the second average molecular weight can be about 1.1-1.6 million Daltons.

The weight ratio of a hyaluronic acid salt of a first average molecular weight to a hyaluronic acid salt of a second average molecular weight can be from about 1:5 to about 5:1 (e.g., about 1:5, about 1:2, about 1:1, about 2:1, about 3:1, about 4: 1, or about 5:1). In some embodiments, the weight ratio of the hyaluronic acid salt of the first average molecular weight to the hyaluronic acid salt of the second average molecular weight can be from about 1:4 to about 4:1 (e.g., from about 1:3 to about 1:1, or from about 1:1 to about 3:1). In certain embodiments, the weight ratio of the hyaluronic acid salt of the first average molecular weight to the hyaluronic acid salt of the second average molecular weight can be from about 1:3 to about 1:1.

In some embodiments, the average molecular weight difference between the hyaluronic acid salt of the first average molecular weight and the hyaluronic acid salt of the second average molecular weight is at least about 0.5 million Daltons (MDa) (e.g., at least about 0.5 MDa, at least about 0.7 MDa, at least about 0.9 MDa, or at least about 1.2 MDa).

The compositions can further comprise a pharmaceutically active ingredient. The pharmaceutically active ingredient can be bone morphogenic protein, tissue growth factors, insulin growth factors, antioxidants, antibiotics, or a combination of thereof.

The compositions can include proteins which can induce the formation of bone and cartilage. For example, bone morphogenic protein, such as BMP-2, BMP-4, BMP-6, and BMP-7.

The compositions can also include an effective amount (either present naturally or intentionally added) of tissue growth factors, e.g., TGF-B. In some embodiments, the composition can have insulin growth factors, e.g., IGF-1. In certain embodiments, the composition can have antioxidants. Exemplary antioxidants include ascorbate, pyruvate, and BHT. In other embodiments, the composition can include antibiotics such as gentamycin and vancomycin.

The compositions can include a mixture of two or more pharmaceutically active ingredients in an amount effective for promoting tissue growth. For example, a mixture of bone morphogenic protein and tissue growth factors, e.g., TGF-β and BMP-2, or a mixture of insulin growth factors and tissue growth factors, e.g., TGF-β and IGF-1

The compositions can also include other therapeutic agents, for example, pain relievers, whether for conditions described herein or some other conditions.

These examples are only for illustrative purpose and any other agents described in literature may be used.

When pharmaceutically active ingredients are used in the compositions, they can be simply mixed with a hyaluronic acid salt and demineralized bone matrix (DBM) in powder form or be blended with a liquid component. Alternatively, the pharmaceutically active ingredients can also be conjugated with a hyaluronic acid salt.

As used herein, demineralized bone matrix (DBM) can be prepared using the methods well known to those skilled in the art. General synthetic methods are found in the literature. See Yee et al. Spine (2003), 28 (21) and Colnot et al. Clinical Orthopaedics and Related Research (2005), (435), 69-78. For example, demineralized bone matrix (DBM) can be prepared by acid extraction of allograft bone, resulting in loss of most of the mineralized component but retention of collagen and non-collagen proteins, including growth factors. DBM can be processed as crushed granules, powder or chips. It can be formulated for use as granules, gels, sponge material or putty and can be freeze-dried for storage. Additionally, DBM can be obtained from commercial sources such as Tissue Banks International (TBI), San Rafael, Calif. or Exactech, Gainesville, Fla.

A hyaluronic acid is a linear long-chain polysaccharide comprising repeating D-glucuronate and N-acetylglucosamine disaccharide units. It can be obtained, for example, either by extraction from animal tissues, such as rooster combs and umbilical cords (Klein, J., & Meyer, F. A., 1983, Biochem. & Biophys. Acta, 755(3), 400-411), or by the removal of hyaluronic acid capsular material from bacterial species, e.g. Streptococcus (Van Brunt, J., 1986, Biotechnology, 4, 780-782).

The hyaluronic acid can further be subjected to gamma irradiation to permit the desired molecular weight reduction to occur (Miller, R. & Shiedlin, A. U.S. Pat. No. 6,383,344). In some embodiments, the hyaluronic acid is essentially water soluble.

The hyaluronic acid can be a modified hyaluronic acid. For example, carboxyl group in the glucuronic acid portion of hyaluronic acid can be converted to a substituted amide group. Suitable substituents of the above substituted amide group may include: an aminoalkyl group (the alkylene chain of which may be substituted with one or more, namely, for example 1 to 8, and preferably 1 to 3 hydroxyl groups.); an amino(polyalkyleneoxy)alkyl group; an amino(polyalkyleneamino)alkyl group; a mercapto(polyalkyleneamino)alkyl group; an acryloyloxyalkyl group; an acryloylaminoalkyl group; and an acryloylamino(polyalkyleneoxy)alkyl group.

In addition, a modified hyaluronic acid can also be cross-linked hyaluronic acid, which normally have a higher molecular weight. A higher molecular weight of hyaluronic acid may be more efficacious due to its enhanced viscoelastic properties.

At physiological pH, the hyaluronic acid may be a salt. In cases where a hyaluronic acid salt is a salt with an inorganic base, such as alkali metal (e.g., lithium, sodium, or potassium) or alkaline earth metal (magnesium or calcium), such salts are easily obtained, for example, by reacting hyaluronic acid with a base containing an alkali metal or alkaline earth metal. The hyaluronic acid salt (e.g., silver salt) can also be prepared by methods as disclosed in U.S. Pat. Nos. 4,784,991 and 5,472,950. In addition, the hyaluronic acid salt can also be purchased from a variety of commercial sources.

The compositions can further include a modified or natural polysaccharide, such as chitosan, chitin, chondroitin sulfate, keratan sulfate, dermatan sulfate, heparin, and heparin sulfate.

The compositions may comprise a natural, recombinant or synthetic protein such as soluble collagen or soluble gelatin or a polyamino acids, such as polylysine.

In some embodiments, the compositions do not include any lipids. In other embodiments, the compositions include less than 12% by weight of lipids (e.g., less than 10%, or less than 8%, or less than 5%, or less than 3%).

Method of Use

The compositions of the present invention can be used to repair cartilage in a subject. The compositions can be administered to the subject at a site of a defect in cartilaginous tissue or a combination of bone and cartilage defect such as in an osteochondral defects. The compositions of the present invention can also be used to repair bone or a defect in other tissues such as meniscus, ligament, tendon, and intervertebral disc annulus. Effective doses will depend on the disease condition being treated as well as by the judgment of the attending clinician depending upon factors such as the severity of the disease, the age, weight and general condition of the patient, and the like.

The composition administered to a patient can be in the form of pharmaceutical compositions (e.g., formable compositions, dry blend compositions, or the plugs) described herein. Hence the dry blend and the plug may be hydrated with a biological fluid prior to use. The pH of the hydrated composition is preferably between 6.5 and 7.8, or preferably between 6.8 and 7.4. The compositions can be sterilized by conventional sterilization techniques, or may be sterile filtered. The compositions can be packaged for use as is, or lyophilized, the lyophilized compositions being combined with a sterile liquid component prior to administration.

The compositions of the invention may be applied directly to the tissue and/or to the site in need of cartilage repair. In some embodiments, the site of treatment in the body may be surgically prepared to remove abnormal tissues, followed by placing the composition of the present invention in the defect area. Alternatively, surgical preparation includes piercing, abrading or drilling into adjacent tissue regions or vascularized regions to create channels for the cells or bone marrow to migrate into the plug or putty. The compositions of the invention can be used to fill an osteochondral defect, or a defect that includes microfractures, or a chondral defect.

### Preparation of the Compositions

In preparing the composition, both DBM and the hyaluronic acid salt can be milled to provide the appropriate particle size prior to combining with the other ingredients, for example, less than 600 microns or less than 850 microns.

DBM and the hyaluronic acid salt may be milled using known milling procedures such as dry or wet milling to obtain a particle size appropriate for a putty or a plug and for other formulation types. Finely divided preparations of the compounds of the invention can be prepared by processes known in the art, for example see International Patent Publication No. WO 2002/000196.

The formable compositions of the present invention can be prepared by mixing a dry blend of hyaluronic acid salt and demineralized bone matrix (DBM) with a liquid component (e.g., PBS). It is an important aspect of the present invention to premix all solid powder components, including the hyaluronic acid salt and DBM, prior to adding the liquid component, e.g., PBS.

The hyaluronic acid salt and demineralized bone matrix (DBM) can be premixed for about 10-24 hours (e.g., about 10 hours, about 12 hours, about 18 hours, about 20 hours, or about 24 hours) to form a solid composition including a homogeneous mixture of DBM and the hyaluronic acid salt. When referring to the composition as homogeneous, the ingredients are typically dispersed evenly throughout the composition. so that the composition can be readily subdivided into equally effective unit dosage forms. It can be understood that when a mixture of at least two hyaluronic acid salts has been used in a composition, all the hyaluronic acid salts will be premixed with DBM. The mixing can be conducted mechanically (e.g., in a high velocity shaker, such as Turbula T2F) until a homogenous powder mixture forms. A liquid component, such as PBS, can be added to the homogeneous powder mixture. The resulting mixture can be mixed, such as in a high velocity shaker, for additional about 12-36 hours (e.g., about 12 hours, about 20 hours, about 24 hours, about 30 hours, or about 36 hours). In some embodiments, a liquid component can be added immediately after mixing DBM and the hyaluronic acid salt(s). In other embodiments, a liquid component can be added just before the intended use.

It is another important aspect of the present invention that two hyaluronic acid salts of different molecular weights can be utilized to create pastes/putties with improved and controlled rheological and biological properties. A number of medically useful substances can be prepared by utilizing teachings of this invention by adding substances to the composition during the mixing process or directly to the final composition. To use a mixture of DBM and the hyaluronic acid salt(s) as a vehicle for in situ drug delivery, drugs can be mixed with powders of DBM and the hyaluronic acid salt(s) and then the compositions can be hydrated. Alternatively, drugs can be mixed with a liquid component, such as PBS and then added to the premixed powder composition. Furthermore, drugs can be conjugated with the hyaluronic acid salt(s) and then be added to the composition. The method of the present invention allows for the preparation of a useable bone putty or paste with at least about 3.5% concentration of the hyaluronic acid salt in the composition. The advantages of using higher concentrations of the hyaluronic acid salt include improved visco-elastic properties of the paste. In addition, the putty or paste prepared by the method of the present invention shows good cohesive properties and minimal adhesion. For example, the putty does not adhere to latex gloves and maintain coherence throughout the handling process, and does not crumble or “pill”. Further, good malleability has also been observed. For example, it can be formed into various shapes (e.g., a sphere) and can hold the intended shape. In some embodiments, the putty is pre-mixed with stem cells or bone marrow cells prior to implantation. In some embodiment the compositions is implanted into the defect by manipulation of a putty into the site. In other embodiments, the composition is implanted into the defect by injection through a needle from a syringe.

The compositions of the invention can also be prepared as a plug. The plug is a bioresorbable scaffold, uniquely designed for the repair and replacement of bone or cartilage. Utilizing combination of HA and DBM according to the invention, this material is designed to be a highly porous scaffold to support bone/cartilage incorporation and remodeling. It is biologically friendly by absorbing fluids and nutrients and uniquely designed to wick the bone marrow into the scaffold.

A plug can offer a more convenient means of scaffold delivery during wet arthroscopic knee surgery. A plug with a porous, osteoconductive structure comprising demineralized bone matrix (DBM) and a hyaluronic acid salt can be obtained by mixing HA with DBM followed by hydration of the powder mixture. For example, a plug can be prepared from a mixture of DBM and a hyaluronic acid salt by lyophilization or other drying process, in custom designed mold to optimize the plug performance and fit into osteochondral defect. A plug is a dry formulation of DBM and a hyaluronic acid salt and therefore will have enhanced stability at room temperature. In some embodiments, the plug can include a plasticizer. Preferred plasticizer can be glycerol or PEG. The plasticizer can be mixed with the powder mixture, e.g., demineralized bone matrix and the hyaluronic acid salt. In some embodiments, the plug is pre-mixed with stem cells or bone marrow cells prior to implantation.

In order that the invention disclosed herein may be more efficiently understood, examples are provided below. It should be understood that these examples are for illustrative purposes only and are not to be construed as limiting the invention in any manner.

### EXAMPLES 1-3

### Preparation of a Bone Putty or Paste

### Example 1

Materials:

Hyaluronic acid salt (HA) powders were purified from the fermentation of Streptococcus zooepidemicus at Genzyme Corporation: MW=1.2 MDa, physical form (fine powder) demineralized bone matrix (DBM) was received from Tissue Banks International (TBI), San Rafael, Calif.

Method 1:

2.4 grams of HA and 21.4 mL of liquid component, PBS, were premixed for 24 hours in high velocity shaker (Turbula T2F, Glen Mills Inc., Clifton, N.J.). After 24 hours of mixing, a homogeneous hydrogel was prepared. At this time, 12 grams of DBM was added. At the end of the process, the composition did not form a putty (HA/DBM dough compositions), but rather showed non uniform mixture that did not incorporate all of the DBM.

Method 2:

12 grams of DBM and 2.4 grams of HA were premixed for 12 hours as powders in a high velocity shaker (Turbula T2F) to make a homogeneous powder mix. At this time, 21.4 mL of liquid component, PBS, was added and the container was returned to a high velocity shaker for additional 24 hours. At the end of the process, homogeneous putty (HA/DBM dough compositions) with good cohesive properties and with minimal adhesion to latex gloves was achieved.

TABLE 1
Compositions of HA/DBM Putties used in Example 1

| HA MW | HA | DBM | PBS | Total |
|---|---|---|---|---|
| 1.2 MDa | 2.4 gram | 12 gram | 21.4 gram | 35.8 gram |

TABLE 2
Calculated Concentrations of HA in the composition and HA
and DBM in the putty in Example 1

| [HA]* w/w | [DBM] w/w | [PBS] w/w |
|---|---|---|
| 6.7% | 33.5% | 59.8% |

*Concentration in the putty (HA + DBM + PBS)

In Example 1, Method 1, putty was prepared by first dissolving HA in PBS and then mixing in DBM to form a putty. Results showed that an inhomogeneous putty was formed. A portion of the DBM could not be incorporated into the putty. In addition, part of the DBM was not wetted with PBS. This is likely due to the competitive absorption of water that occurs between HA and DBM. In Example 1, Method 2, a putty was prepared by first uniformly dispersing the HA and DBM powders and then adding the PBS. This resulted in a uniform and cohesive malleable putty.

### Example 2

Materials:

Hyaluronic acid salt (HA) powder was purified from the fermentation of Streptococcus zooepidemicus at Genzyme Corporation: MW=1.2 MDa, physical form (fine powder). HA powder was purified from the fermentation of Streptococcus zooepidemicus at Genzyme Corporation and then sent for gamma irradiation to reduce the molecular weight to 500 kDa (Miller, R. & Shieldlin, A. U.S. Pat. No. 6,383,344), physical form (fine powder). Demineralized bone matrix was received from Tissue Banks International (TBI) San Rafael, Calif.

Method:

12 grams of DBM and 2 grams of HA molecular weight 1.2 MDa and 1 gram of HA molecular weight 500 kDa were premixed for 12 hours as powders in a high velocity shaker (Turbula T2F) to make a homogeneous powder mix. At this time, 21.4 mL of liquid component PBS was added and the container was returned to a high velocity shaker for additional 24 hours. At the end of the process, putty (HA/DBM dough compositions) with good cohesive properties and with minimal adhesion to latex gloves was achieved.

TABLE 3
Compositions of HA/DBM putties used in Example 2

| HA MW = 0.5 MDa | HA MW = 1.2 MDa | DBM | PBS | Total |
|---|---|---|---|---|
| 1 gram | 2 gram | 12 gram | 21.4 gram | 36.4 gram |

TABLE 4
Calculated Concentrations of HA in the composition
and HA and DBM in the putty in Example 2

| [HA]** w/w | [DBM] w/w | [PBS] w/w |
|---|---|---|
| 8.24% | 33% | 58.76% |

**Concentration in the putty (HA + DBM + PBS)

In Example 2, putty with good cohesive properties and with minimal adhesion to latex gloves was achieved using 14% HA in the composition. In addition, this composition used two HA molecular weights, 0.5 and 1.2 MDa HA. Mixing two molecular weight HA components allows for the manipulation of rheological properties.

### Example 3

Materials: Hyaluronic acid salt (HA) powder was purified from the fermentation of Streptococcus zooepidemicus at Genzyme Corporation: MW=1.2 MDa, physical form (fine powder). HA powder was purified from the fermentation of Streptococcus zooepidemicus at Genzyme Corporation and then sent for gamma irradiation to reduce the molecular weight to 500 kDa (Miller, R. & Shieldlin, A. U.S. Pat. No. 6,383,344), physical form (fine powder). Demineralized bone matrix was received from Tissue Banks International (TBI) San Rafael, Calif.

Method:

9 grams of DBM, 1.9 grams of HA molecular weight 1.2 MDa and 0.85 gram of HA molecular weight 500 kDa were premixed for 12 hours as powders in a high velocity shaker (Turbula T2F) to make a homogeneous powder mix. 0.9 gram (1.4 mL) of mixed powders was placed in a weight boat and 1.5 grams of PBS was added. After this addition, the powder and PBS were mixed with the spatula until a homogeneous putty was achieved (30 second).

TABLE 5
Compositions of HA/DBM powder used in Example 3

| | | | [HA] | [DBM] |
|---|---|---|---|---|
| HA MW = 0.5 MDa | HA MW = 1.2 MDa | DBM | w/w | w/w |
| 0.85 gram | 1.9 gram | 9 gram | 23% | 77% |

TABLE 6
Compositions of HA/DBM putty used in Example 3

| HA | DBM | PBS | [HA] w/w | [DBM] w/w |
|---|---|---|---|---|
| 0.21 gram | 0.69 gram | 15 gram | 8.75% | 29% |

In Example 3, putty with good cohesive properties and with minimal adhesion to latex gloves was achieved using 23% HA in the HAIDBM mix. After the powders were mixed, PBS was added as needed to create a paste with desired rheological properties. In addition, this composition used two HA molecular weights, 0.5 and 1.2 MDa HA. Mixing two molecular weights HA components allows for the manipulation of rheological properties.

### Example 4

### Extrusion Force and Measurements of Extrusion Force for Acceptable Putties

Additional test to characterize mechanical properties of HA/DBM were developed. The Instron Syringe Extrusion Force is a test used to characterize properties of putty. In this test, force required pushing material through a 3 mL syringe with a diameter of 8.6 mm and 15 gauge 1-½ needle, in constant rate is measured. The extrusion force was measured during compression at the rate 0.5 mm/sec until plateau was reached. (see FIG. 1). Five HA/DBM formulations were measured and compared to DBX™ (commercially available DBM from Synthes, Pa.).

TABLE 7

| Lot # | [HA] | [DBM] | Force [N] |
|---|---|---|---|
| A | 6% 70 kDa; 6% 1.6 MDa | 31% | 22.9 ± 0.7 |
| B | 4% 0.5 MDa; 4% 1.2 MDa | 31% | 21.3 ± 0.7 |
| C | 6% 1.2 MDa | 31% | 18.6 ± 0.7 |
| D | 5% 3 MDa | 31% | 14.0 ± 1.4 |
| E | 8% 660 kDa | 31% | 25.8 ± 0.4 |
| DBX ™ | 2.8% 1 MDa | 31% | 16.2 ± 1.4 |

### Example 5

### Absorption of HA-DBM Plug Formulations

HA-DBM putty was prepared as described in Example 2 or by manually mixing HA-DBM powders with liquid component PBS in two syringes with luer-lock syringe connector. The homogeneous putty was loaded into Teflon molds with desired dimensions. The shaped putty was frozen inside the mold at −80° C. for at least 4 hours prior to freeze drying overnight. The resulting HA-DBM dry plugs were removed from the Teflon molds.

To optimize the ability of a plug to wick the bone marrow into the scaffold, absorption of the different plug formulations was tested in following method.

Method: Both ends of a 1 cc syringe were cut off and a 105-149 um polypropylene mesh was adhered to one end of the syringe. One HA-DBM plug was placed in each syringe and the initial height of the plug was recorded. The syringe was placed in a 15mL conical tube and dye in PBS was placed in the tube to just cover the bottom end of the syringe (end with filter attached). Samples were placed at 37 ° C. Dye migration into each plug was recorded over a period of 7 days. Time to 100% dye absorption into the plug was used to compare absorption rates for various formulations.

TABLE 8

| Formulation | Formulation | Time to 100% | |
|---|---|---|---|
| [DBM] | [HA], HA MW | Absorption | |
| 97% | 3% 450 kDa | <30 | min. |
| 94% | 6% 450 kDa | 4-5 | hr. |
| 88% | 12% 450 kDa | >24 | hr. |

From this data, it appears that a slower rate of absorption is attributed to higher molecular weight HA and/or a higher concentration of HA.

### Example 6

### Swelling of HA-DBM Plug Formulations

To maintain maximum concentration of DBM in vivo, plugs with minimal swelling are desirable. To measure the swelling, different plug formulations were tested in the following method:

Method: Both ends of a 1 cc syringe were cut off and a 5 um polypropylene mesh was adhered to one end of the syringe. One HA-DBM plug was placed in each syringe and the initial height of the plug was recorded. The syringe was placed in a 15 mL conical tube and PBS was placed in the tube and inside the syringe. Samples were placed at 37° C. The height of each plug was recorded over a period of 72 hours. The difference in height of the plug was used to calculate percent swelling.

TABLE 9

| Formulation | Formulation | Percent Swell | Percent Swell | Percent Swell |
|---|---|---|---|---|
| [DBM] | [HA], HA MW | at T = 2 hr | at T = 24 hr | at T = 72 hr |
| 94% | 6% 200 kDa | 29.0 ± 3.5 | 34.0 ± 0.0 | 34.0 ± 7.1 |
| 94% | 6% 450 KDa | 37.2 ± 1.2 | 39.7 ± 11.2 | 38.1 ± 11.3 |
| 88% | 12% 450 kDa | 16.3 ± 3.8 | 35.0 ± 4.4 | 44.3 ± 4.7 |
| 83% | 17% 450 kDa | 6.8 ± 3.2 | 31.8 ± 0.0 | 36.4 ± 0.0 |
| 83% | 17% 1.6 MDa | 11.4 ± 3.2 | 34.1 ± 3.2 | 81.8 ± 0.0 |

From this data, it appears that an increase in swelling is attributed to higher molecular weight HA. The concentration of HA may also affect the swelling of HA-DBM plugs.

### Example 7

### Arthroscopic Delivery Evaluation of HA/DBM Plug Formulations

The ability of the plug to retain integrity during handling was tested in following methods:

Method: A synthetic, articulating knee joint was used to simulate arthroscopic delivery conditions. Standard arthroscopic equipment was used to create access ports in the knee and to visualize the procedure within the joint capsule. Standard fluid management equipment was used to control flow rates within the joint capsule and to distend the joint capsule as needed. Osteochondral defects were created on the medial and femoral condyles. HA/DBM plugs were loaded into a tube and plunger device. The distal end of the device was introduced into the joint space through a cannula and aligned with the defect. At this point, constant fluid flow was initiated and the HA/DBM plug was implanted into the osteochondral defect by advancing the plunger. Plugs were evaluated for integrity and retention properties under flowing conditions during and after implantation (5-60 minutes).

Test results (Table 10) indicated that plug formulations with an HA concentration of 6% and a molecular weight of 200,000 Daltons were displaced from the application site under constant fluid flow. In contrast, plugs with an HA concentration of 6% or higher and at least a molecular weight of 450,000 Daltons were successfully retained in the application site under constant fluid flow.

TABLE 10

| | Formulation | Formulation | |
|---|---|---|---|
| Entry | [DBM] | [HA], HA MW | Arthroscopic Delivery Results |
| A | 94% | 6% 200 kDa | Fail - implant displaced from site |
| B | 94% | 6% 450 kDa | Pass - implant retained at site |
| C | 88% | 12% 450 kDa | Pass - implant retained at site |
| D | 79% | 21% 1.6 MDa | Pass - implant retained at site |

### Example 8

### Unconfined Compression Testing of HA/DBM Plug Formulations

Method:

An Instron mechanical test instrument was used to measure the unconfined compressive properties of dry HA/DBM plugs. At the start of each test, the plugs were preloaded to 1 N at a rate of 1 mm/min. After preloading, the plugs as described in Table 11 were compressed at a rate of 1 mm/min until a compressive strain of 13% was achieved. Values for compressive stress (at 10% strain) and modulus (between 5 and 10% strain) were calculated from the stress-strain curve.

TABLE 11

| | | | Comp. Stress | Modulus | Modulus |
|---|---|---|---|---|---|
| | Height | Diameter | (10%) | (Automatic) | (5-10%) |
| Specimen | (mm) | (mm) | (MPa) | (MPa) | (MPa) |
| 1 | 9.49 | 4.76 | 1.57 | 32.81 | 11.19 |
| 2 | 9.61 | 4.74 | 1.64 | 30.78 | 12.37 |
| 3 | 9.87 | 4.75 | 1.69 | 26.50 | 13.51 |
| Mean | 9.66 | 4.75 | 1.63 | 30.03 | 12.36 |
| Std. Dev. | 0.19 | 0.01 | 0.06 | 3.22 | 1.16 |

Specimens 1-3 exhibit suitable compression stress. These results are based on the testing of 3 individual plugs comprising 12% HA (450k Da)+88% DBM. This formulation passed the arthroscopic evaluation as shown in Table 10, Entry C.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Various modifications of the invention, in addition to those described herein, will be apparent to those skilled in the art from the foregoing description. Such modifications are also intended to fall within the scope of the appended claims.

1. A formable composition comprising from about 25% to about 40% by weight of demineralized bone matrix, from about 3.5% to about 25% by weight of a hyaluronic acid salt, and from about 40% to about 72% by weight of a biologically compatible liquid.

2. (canceled)

3. (canceled)

4. (canceled)

5. The composition of claim 1 further comprising a rheology modifier.

6. (canceled)

7. (canceled)

8. (canceled)

9. (canceled)

10. The composition of claim 1 wherein the hyaluronic acid salt has an average molecular weight of at least about 500,000 Daltons.

11. The composition of claim 1 comprising a mixture of at least two hyaluronic acid salts.

12. (canceled)

13. (canceled)

14. (canceled)

15. (canceled)

16. (canceled)

17. The composition of claim 1 further characterized in that, when subject to the Instron Syringe Extrusion Force (ISEF) test, the composition exhibits an extrusion force of from about 12.0 Newtons to about 30.0 Newtons.

18. (canceled)

19. (canceled)

20. The composition of claim 1 further comprising a pharmaceutically active ingredient.

21. The composition of claim 20 wherein the pharmaceutically active ingredient is bone morphogenic protein, tissue growth factors, insulin growth factors, antioxidants, antibiotics, or combinations of growth factors.

22. The composition of claim 20 wherein the pharmaceutically active ingredient is selected from BMP-2, BMP-4, BMP-6, BMP-7, TGF-B, IGF-I, ascorbate, pyruvate, BHT, gentamycin, vancomycin, the combination of TGF-β and BMP-2, and the combination of TGF-β and IGF-I.

23. The composition of claim 20 wherein the pharmaceutically active ingredient is conjugated with the hyaluronic acid salt.

24. A method of repairing cartilage in a subject comprising administering to a subject at a site of a defect in cartilaginous tissue an effective amount of a composition, the composition comprising demineralized bone matrix and a hyaluronic acid salt.

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. A dry blend composition comprising from about 60% to about 92% by weight of demineralized bone matrix, from about 3.5% to about 38% by weight of a hyaluronic acid salt, and from about 3% to about 10% by weight of a biologically compatible liquid.

77. (canceled)

78. The composition of claim 76 wherein the hyaluronic acid salt has an average molecular weight of at least about 200,000 Daltons.

79. The composition of claim 76 comprising a mixture of at least two hyaluroinc acid salts.

80. (canceled)

81. (canceled)

82. (canceled)

83. The composition of claim 76 further comprising a pharmaceutically active ingredient.

84. The composition of claim 83 wherein the pharmaceutically active ingredient is bone morphogenic protein, tissue growth factors, insulin growth factors, antioxidants, antibiotics, or combinations of growth factors.

85. The composition of claim 83 wherein the pharmaceutically active ingredient is selected from BMP-2, BMP-4, BMP-6, BMP-7, TGF-B, IGF-I, ascorbate, pyruvate, BHT, gentamycin, vancomycin, the combination of TGF-β and BMP-2, and the combination of TGF-β and IGF-I.

86. The composition of claim 83 wherein the pharmaceutically active ingredient is conjugated with the hyaluronic acid salt.

87. A plug comprising from about 25% to about 88% by weight of demineralized bone matrix and from about 3.5% to about 38% by weight of a hyaluronic acid salt and from about 3% to about 20% by weight of a biologically compatible liquid.

88. (canceled)

89. (canceled)

90. The plug of claim 87 wherein the powders further comprise a plastizer.

91. (canceled)

92. (canceled)

93. (canceled)

94. (canceled)

95. (canceled)

96. The plug of claim 87 wherein the hyaluronic acid salt has an average molecular weight of at least about 200,000 Daltons.

97. The plug of claim 87 comprising a mixture of at least two hyaluronic acid salts.

98. (canceled)

99. (canceled)

100. (canceled)

101. (canceled)

102. (canceled)

103. The plug of claim 87 wherein the powders further comprise a pharmaceutically active ingredient.

104. The plug of claim 103 wherein the pharmaceutically active ingredient is bone morphogenic protein, tissue growth factors, insulin growth factors, antioxidants, antibiotics, or combinations of growth factors.

105. The plug of claim 103 wherein the pharmaceutically active ingredient is selected from BMP-2, BMP-4, BMP-6, BMP-7, TGF-B, IGF-I, ascorbate, pyruvate, BHT, gentamycin, vancomycin, the combination of TGF-β and BMP-2, and the combination of TGF-β and IGF-I.

106. The plug of claim 103 wherein the pharmaceutically active ingredient is conjugated with the hyaluronic acid salt.

107. The plug of claim 87 characterized in that the plug exhibits an unconfined Compression Stress of at least about 1.55 MPa.

108. A method of forming a plug comprising a) providing powders of demineralized bone matrix and a hyaluronic acid salt, b) mixing the powders, c) adding a liquid component to form a putty like material, d) placing the putty in a mold, and e) drying the shaped putty to form a plug.

109. (canceled)

110. (canceled)

111. (canceled)

112. (canceled)

113. (canceled)

114. (canceled)

115. (canceled)

116. (canceled)

117. (canceled)

118. (canceled)

119. (canceled)

120. (canceled)

121. (canceled)

122. (canceled)

123. (canceled)

124. (canceled)

125. (canceled)

126. (canceled)

127. (canceled)

128. (canceled)

129. (canceled)

130. The method of claim 108 wherein the plug is further conditioned to achieve a moisture content of about 3-20% by weight.

131. The method of claim 130 wherein the moisture content is from about 5% to about 10% by weight.
